(19) United States
(12) Patent Application Publication
(10) Pub. No.: US 2007/0212310 A1
(43) Pub. Date: Sep. 13, 2007

(54) Antimicrobial smoke flavor for oral microflora control

(75) Inventors: Yawen Wu, Roscoe, IL (US); John Bedford, Cam (GB); Kevin Riley, Hackettstown, NJ (US)

Correspondence Address: JENKINS, WILSON, TAYLOR & HUNT, P. A., SUITE 1200, UNIVERSITY TOWER, 3100 TOWER BOULEVARD, DURHAM, NC, 27707, US

(73) Assignee: Mastertaste Inc., Brentwood, TN (US)

(21) Appl. No.: 11/707,255

(22) Filed: Feb. 14, 2007

Related U.S. Application Data
(60) Provisional application No. 60/773,075, filed on Feb. 14, 2006.

(51) Int. Cl. A61K 9/68; A61K 9/70; A61K 36/185

(52) U.S. Cl. 424/48; 424/443; 424/725.1

(57) ABSTRACT

The presently disclosed subject matter provides compositions and methods for inhibiting the growth of oral microbes in a subject. Particularly, the methods include applying an oral composition comprising an oral care delivery agent and an antimicrobial agent comprising an effective amount of a low flavor antimicrobial liquid smoke derivative to the oral cavity of the subject for a time sufficient to inhibit growth of a microorganism in the oral cavity of the subject. Compositions that include low flavor antimicrobial liquid smoke derivatives that can be employed in the disclosed methods are also provided.

### RELATED APPLICATIONS

This application claims the benefit of U.S. Provisional Patent Application Ser. No. 60/773,075, filed Feb. 14, 2006, the disclosure of which is incorporated herein by reference in its entirety.

### TECHNICAL FIELD

The presently disclosed subject matter relates to methods and compositions for inhibiting the growth of oral microbes in a subject. More particularly, the presently disclosed subject matter relates to methods for treating food products and/or oral care products with low flavor anti-microbial derivatives of liquid smoke that do not impart undesirable smoky flavors to the food products and/or oral care products such that when the food products and/or oral care products are chewed or otherwise incubated in the mouth of a subject the growth of microorganisms in the mouth of the subject is inhibited.

### BACKGROUND

Oral diseases such as tooth decay and gum disease continue to be a global health problem, especially in poorer countries. In fact, in the World Oral Health Report 2003, the World Health Organization disclosed that an estimated five billion people worldwide had experienced dental caries. This Report further stated that dental caries affect a very large percentage of both schoolchildren and adults.

Oral diseases result in pain and suffering, impaired function, and reduced quality of life, and are estimated to account for between 5-10% of health care costs in industrialized countries (see “World Oral Health Report 2003, available from the World Health Organization). Thus, there continues to be considerable demand for products that can be employed to reduce the growth of oral bacteria. Additionally, oral bacteria can generate various compounds that contribute to bad breath, which while not negatively impacting one's health per se, can be a source of embarrassment and a reduced quality of life.

Fortunately, proper oral care can reduce or eliminate many of the consequences of the presence of oral microbes. However, since oral cleansing and breath freshening can be difficult or inconvenient at times, there is a continuing need for new compositions that can be conveniently employed to reduce the growth of oral microbes, particularly those that are known to cause conditions such as tooth decay, gum disease, and bad breath.

### SUMMARY

The presently disclosed subject matter provides methods and compositions for inhibiting the growth of oral microbes in a subject, and for providing aesthetic and health benefits therefrom.

In some embodiments, the presently disclosed subject matter provides oral compositions for freshening the breath of a consumer of the oral composition. In some embodiments, the oral compositions comprise (a) an oral delivery agent; and (b) an antimicrobial agent comprising an effective amount of a low flavor antimicrobial liquid smoke derivative. In some embodiments, the oral delivery agent is selected from the group consisting of a chewing gum, an edible film, a confectionary, a dentifrice, a lozenge, a mouthwash, and a mouth spray. In some embodiments, the low flavor antimicrobial liquid smoke derivative comprises (a) titratable acidity in a concentration of 0 to about 6% weight per unit volume (w/v); (b) at least about 3% weight per unit volume (w/v) carbonyl; and (c) phenolics in a concentration of less than about 2.5 mg/ml. In some embodiments, the low flavor antimicrobial liquid smoke derivative comprises carbonyl in a concentration of about 8.0 to about 12.0% weight per unit volume (w/v), and a pH of less than about 6.0. In some embodiments, the low flavor antimicrobial liquid smoke derivative comprises carbonyl in a concentration of about 3.0 to about 8.0% weight per unit volume (w/v), phenolics in a concentration of less than about 1 mg/ml, and a pH of about 2.0 to about 5.5. In some embodiments, the low flavor antimicrobial liquid smoke derivative comprises carbonyl of at least 10% weight per unit volume (w/v). In some embodiments, the low flavor antimicrobial liquid smoke derivative is produced by processing liquid smoke through an evaporator to separate and condense the low boiling elements thereof to produce the derivative of liquid smoke.

The oral compositions of the presently disclosed subject matter can be prepared in various forms for use by consumers. In some embodiments, the oral compositions of the presently disclosed subject matter are provided in the form of a chewing gum composition. In some embodiments, the chewing gum composition comprises (a) a water soluble bulk portion; (b) at least one flavoring agent; (c) a gum base portion; and (d) an effective amount of a low flavor antimicrobial liquid smoke derivative. In some embodiments, the oral compositions of the presently disclosed subject matter are provided in the form of a confectionary composition. In some embodiments, the confectionary composition comprises (a) at least one sugar, sugar alcohol, sugar substitute, or a combination thereof; and (b) an effective amount of a low flavor antimicrobial liquid smoke derivative. In some embodiments, the oral compositions of the presently disclosed subject matter are provided in the form of an edible film composition. In some embodiments, the edible film composition comprises (a) an effective amount of a film forming agent; and (b) an effective amount of a low flavor antimicrobial liquid smoke derivative.

The presently disclosed subject matter also provides methods for oral cleansing. In some embodiments, the methods for oral cleansing comprise applying an oral composition to the oral cavity of a subject, wherein the oral composition comprises (a) an oral delivery agent; and (b) an effective amount of a low flavor antimicrobial liquid smoke derivative. In some embodiments, the low flavor antimicrobial liquid smoke derivative comprises (a) titratable acidity in a concentration of 0 to about 6% weight per unit volume (w/v); (b) at least about 3% weight per unit volume (w/v) carbonyl; and (c) phenolics in a concentration of less than about 2.5 mg/ml. In some embodiments, the low flavor antimicrobial liquid smoke derivative comprises carbonyl in a concentration of about 8.0 to about 12.0% weight per unit volume (w/v), and a pH of less than about 6.0. In some embodiments, the low flavor antimicrobial liquid smoke derivative comprises carbonyl in a concentration of about 3.0 to about 8.0% weight per unit volume (w/v), phenolics in a concentration of less than about 1 mg/ml, and a pH of about 2.0 to about 5.5. In some embodiments, the low flavor antimicrobial liquid smoke derivative comprises carbonyl of at least 10% weight per unit volume (w/v). In some embodiments, the low flavor antimicrobial liquid smoke derivative is produced by processing liquid smoke through an evaporator to separate and condense the low boiling elements thereof to produce the derivative of liquid smoke. In some embodiments, the oral composition further comprises at least one flavoring agent.

The presently disclosed subject matter also provides methods for inhibiting growth of a microorganism in the oral cavity of a subject. In some embodiments, the methods comprise applying an oral composition comprising an oral care delivery agent and an antimicrobial agent comprising an effective amount of a low flavor antimicrobial liquid smoke derivative to the oral cavity of the subject for a time sufficient to inhibit growth of a microorganism in the oral cavity of the subject. In some embodiments, the microorganism is selected from the group consisting of Streptococcus mutans, Fusobacterium nucleatum, and Porphrymonas gingivalis. In some embodiments, the oral delivery agent is selected from the group consisting of a chewing gum, an edible film, a confectionary, a dentifrice, a lozenge, a mouthwash, and a mouth spray. In some embodiments, the derivative of liquid smoke comprises (a) titratable acidity in a concentration of 0 to about 6% weight per unit volume (w/v); (b) at least about 3% weight per unit volume (w/v) carbonyl; and (c) phenolics in a concentration of less than about 2.5 mg/ml. In some embodiments, the derivative of liquid smoke comprises carbonyl in a concentration of about 8.0 to about 12.0% weight per unit volume (w/v), and a pH of less than about 6.0. In some embodiments, the derivative of liquid smoke comprises carbonyl in a concentration of about 3.0 to about 8.0% weight per unit volume (w/v), phenolics in a concentration of less than about 1 mg/ml, and a pH of about 2.0 to about 5.5. In some embodiments, the derivative of liquid smoke comprises carbonyl of at least 10% weight per unit volume (w/v). In some embodiments, the derivative of liquid smoke is produced by processing liquid smoke through an evaporator to separate and condense the low boiling elements thereof to produce the derivative of liquid smoke. In some embodiments, the oral composition further comprises at least one flavoring agent.

Thus, it is an object of the presently disclosed subject matter to provide compositions and methods for inhibiting or reducing the growth of oral microbes in a subject.

An object of the presently disclosed subject matter having been stated hereinabove, and which is achieved in whole or in part by the presently disclosed subject matter, other objects will become evident as the description proceeds.

### DETAILED DESCRIPTION

I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

Following long-standing patent law tradition, the terms “a”, “an”, and “the” are meant to refer to one or more as used herein, including the claims. For example, the phrase “a cell” can refer to one or more cells. Also as used herein, the term “another” can refer to at least a second or more.

The term “about”, as used herein when referring to a measurable value such as an amount of weight, time, dose (e.g., a number of cells), etc., is meant to encompass variations of in some embodiments ±20%, in some embodiments ±10%, in some embodiments, ±5%, in some embodiments ±1%, and in some embodiments ±0.1% from the specified amount, as such variations are appropriate to perform the disclosed methods.

The use of the term “or” in the claims is used to mean “and/or” unless explicitly indicated to refer to alternatives only or the alternatives are mutually exclusive.

As used herein, the term “subject” refers to any organism for which application of the presently disclosed subject matter would be desirable. A subject in its many embodiments is desirably a human subject, although it is to be understood that the principles of the presently disclosed subject matter indicate that the presently disclosed subject matter is effective with respect to all vertebrate species, including mammals, which are intended to be included in the term “subject”. Moreover, a mammal is understood to include any mammalian species for which a reduction in the number and/or type of microbes present in the oral cavity of the mammal would be desirable, particularly agricultural, domestic, and companion mammalian species.

As used herein, the phrase “liquid smoke (LS)” refers to a solution comprising liquid reagents capable of imparting a smoky hue and/or coloration and/or smoky flavor to a food product exposed to a liquid or vapor phase of the solution. The use of liquid smoke in lieu of wood smoke is now conventional in meat processing and can be more fully appreciated with reference to representative U.S. Pat. Nos. 3,873,741; 4,250,199; 4,298,435; and 5,043,174.

LS has also been found to possess antimicrobial activity. For example, U.S. Pat. No. 5,043,174 discloses that treatment of hot dogs with LS (ZESTI-SMOKE Code 10, available from Mastertaste of Crossville, Tenn., United States of America) can prevent post-processing re-inoculation with Listeria monocytogenes. However, the LS used imparts significant smoky flavor on the treated food, which can be undesirable under certain circumstances. Thus, it would be advantageous to generate derivatives of liquid smoke that retain antimicrobial activity yet impart no smoky flavor to food products or oral care products during the treatment stage.

As used herein, the phrase “derivative of liquid smoke (DLS)” refers to a derivative of liquid smoke that has characteristics that are appropriate for a given use. DLSs are typically fractions of a liquid smoke that are prepared, for example, by processing a conventional liquid smoke through an evaporator, which separates and condenses the low boiling elements of the liquid smoke to produce the derivative of liquid smoke solution. Accordingly, the phrases “derivative of liquid smoke”, “derivative”, “liquid smoke fraction”, and “fraction” are used interchangeably herein and refer to a component of a liquid smoke that has been isolated from the liquid smoke itself, either with or without subsequent additional preparation and/or modification steps. In some embodiments, a DLS is characterized by antimicrobial activity and does not impart smoky flavors to a food product or an oral care product when the food product is treated with the DLS.

As used herein, the phrase “antimicrobial activity” refers generally to an activity of a liquid smoke or a derivative of a liquid smoke that results in either the killing of a microorganism (including, but not limited to microbicidal and microbiolytic activities) and/or the inhibition of the growth of a microorganism (including, but not limited to a microbiostatic activity). With respect to an inhibition of microbial growth, the term “antimicrobial activity” is intended to encompass both total inhibition (i.e. the microorganism does not grow at all or at an undetectable rate in the presence of the DLS) and partial inhibition, the latter being characterized by either a delay in the initiation of growth of the microorganism or a reduction in the rate at which the microorganism grows, or both.

II. Preparation of Liquid Smoke Derivatives from Liquid Smoke

As is well known to those skilled in the art, liquid smoke compositions obtained from pyrolysis of hardwood sawdust contain constituents primarily from the thermal degradation of cellulose, hemicellulose, and lignin. More particularly, the liquid smoke compositions contain a wide array of over 400 chemical compounds, and hence, liquid smoke compositions are characterized by their content of certain classes of compounds, namely, acids (% titratable acidity, determined using the method disclosed in U.S. Pat. No. 6,214,395), phenolics, and carbonyls.

The acids are preservatives and pH control agents. Commercial liquid smoke compositions typically have a pH under about 2.5, and more typically under about 2.3, and a % titratable acidity by volume from about 3% to about 18%. The phenolics give a smoky flavor, and also aroma, to liquid smoke compositions, which typically have a phenolics content from about 10 to about 45, and more typically, from about 14 to about 30 mg/ml. The carbonyls impart the brown color-forming capacity to liquid smoke compositions. The phenolics and the carbonyls can be measured as described in U.S. Pat. No. 4,431,032, which describes techniques for the removal of an undesirable tar component from liquid smoke compositions. It is noted that the acids and carbonyls are secondary in contributing to the smoky flavor of liquid smoke compositions.

Mastertaste of Crossville, Tenn., United States of America, is a manufacturer of various liquid smokes. Exemplary liquid smokes include ZESTI-SMOKE Code 10 and ZESTI-SMOKE Code V. The specifications of these liquid smokes are given in Table 1:

TABLE 1
Compositions of Exemplary Liquid Smokes

| | Code 10 | Code V |
|---|---|---|
| Acidity (% w/v) | 10.5-11.0 | 6.8-7.8 |
| Staining Index | 69-80 | None |
| Carbonyl level (g/100 mL) | 15-25 | 2.0-7.0 |
| Phenolic level (mg/mL) | 12-22 | 1.0-4.0 |
| Specific gravity (25° C.) | 1.068-1.079 | 1.005-1.015 |
| Density (lbs/gal) | 8.90-8.99 | 8.37-8.46 |
| pH | 2-3 | 2.0-2.4 |
| Color | Amber | Amber |

The ZESTI-SMOKE Code V fraction set forth hereinabove can be produced as a derivative or secondary product of ZESTI-SMOKE Code 10. The Code 10 can be processed through a separator (for example, an AVP evaporator) by feeding the Code 10 as a feed stock which is first heated to remove low boiling acids from the top of the evaporator and then is condensed into Code V as a secondary product. This process also yields a concentrated liquid smoke that has higher percent acid, staining index, carbonyl and phenolic levels, specific gravity, density, and darker color than conventional liquid smoke, and that is sold under the trademark SUPERSMOKE™ by Mastertaste of Crossville, Tenn., United States of America, for a variety of end uses.

The Code V derivative is a low pH, low flavor, low or no stain product. In some embodiments, Code V is then treated with a suitable pH adjustment agent, such as sodium bicarbonate, sodium carbonate, sodium hydroxide, or potassium hydroxide, in order to bring the pH up to at least about 5.0. The pH can be brought up to as high as about 7.0. In some embodiments, the pH ranges from about 5.0 to about 6.0. This pH-adjusted material can be further modified to produce a derivative of liquid smoke as disclosed herein.

In some embodiments, the Code V derivative is first treated with carbon in accordance with the method disclosed in U.S. Pat. No. 5,637,339 to Moeller. This removes phenolics. The resultant is then treated with the suitable pH adjustment agent. Optionally, the pH adjustment can be performed prior to the carbon treatment. This carbon treated, pH adjusted material can also be used as a starting material for producing a derivative of liquid smoke as used herein.

In some embodiments, the sawdust is delignified prior to pyrolysis to produce low flavor products. Various of these products and derivatives can be blended together to produce a range of carbonyl levels. They can be carbon treated to substantially reduce phenolics. They can also be treated with neutralizing agents to adjust pH/acidity.

In the Examples presented below, the antimicrobial activities of various derivatives of liquid smoke were tested versus certain strains of bacteria that are known to be related to oral diseases.

III. Compositions and Methods for Inhibiting Growth of Oral Microbes

III.A. Compositions

The use of chewing gums, confections, thin films, and other compositions as vehicles for delivering components to the oral cavity has been known to provide oral benefits such as breath freshening and bactericidal properties. Such compositions provide a consumer with a convenient and inexpensive method for maintaining oral health and fresh breath throughout the course of the day.

The presently disclosed subject matter relates in some embodiments to oral compositions comprising natural low-flavor antimicrobials derived from smoke flavors to control oral microflora by incorporating the natural low-flavor antimicrobials into food and oral care products including, but not limited to confectionary products, chewing gums, edible films, toothpaste, and mouthwash. Additionally, the presently disclosed methods can be extended to food products that are appropriate for non-human species (e.g., pet foods). The disclosed antimicrobials have demonstrated strong efficacy against oral microorganisms that cause bad breath and gum diseases in humans, yet due to their low flavor impact properties impart little or no sensory changes to the products to which the agents are applied. Additionally, unlike chemical or synthetic antimicrobial agents that must be declared distinctively, the antimicrobials described in the presently disclosed subject matter can be labeled as “natural flavor” on the ingredient statement of the treated products.

In some embodiments of the presently disclosed subject matter, an oral composition is provided comprising an effective amount of a low flavor antimicrobial liquid smoke derivative. As used herein, the term “effective amount” refers to an amount of a low flavor antimicrobial liquid smoke derivative that is present in an oral formulation that when consumed or otherwise incubated in the oral cavity of a subject results in a measurable decrease in the number, viability, and/or growth rate of a microorganism present within the subject's oral cavity. It is understood that the amount of time during which the oral composition is intended to remain in the oral cavity of the subject can vary, and is in some embodiments that amount of time for which a subject would normally maintain a like formulation (e.g., a chewing gum, an edible film, a confectionary, a dentifrice, a lozenge, a mouthwash, or a mouth spray) in the subject's oral cavity. It is thus understood that the amount of time can be measured in seconds (e.g., 5, 10, 15, 20, 30, 45, or 60 seconds, as well as all time points in between), minutes (e.g., 1, 2, 3, 4, 5, 6, 7, 8, 9, 10, 11, 12, 13, 14, 15, 20, 25, 30, 35, 40, 45, 50, 55, or 60 minutes, as well as all time points in between), or longer.

In some embodiments, a low flavor antimicrobial liquid smoke derivative comprises (a) titratable acidity in a concentration of 0 to about 6% weight per unit volume (w/v); (b) at least about 3% weight per unit volume (w/v) carbonyl; and (c) phenolics in a concentration of less than about 2.5 mg/ml. In some embodiments, a low flavor antimicrobial liquid smoke derivative comprises carbonyl in a concentration of about 8.0 to about 12.0% weight per unit volume (w/v), and a pH of less than about 6.0. In some embodiments, a low flavor antimicrobial liquid smoke derivative comprises carbonyl in a concentration of about 3.0 to about 8.0% weight per unit volume (w/v), phenolics in a concentration of less than about 1 mg/ml, and a pH of about 2.0 to about 5.5. In some embodiments, a low flavor antimicrobial liquid smoke derivative comprises carbonyl of at least 10% weight per unit volume (w/v).

In some embodiments, the low flavor antimicrobial liquid smoke derivative is present in the oral composition in the maximum amount that does not negatively effect the flavor properties of the oral delivery agent (e.g., the chewing gum, the edible film, the confectionary, the dentifrice, the lozenge, the mouthwash, or the mouth spray). In some embodiments, the low flavor antimicrobial liquid smoke derivative can be present in the oral delivery agent in some embodiments in an amount up to 50% by weight of the oral delivery agent, in some embodiments in an amount up to 25% by weight of the oral delivery agent, in some embodiments in an amount up to 20% by weight of the oral delivery agent, in some embodiments in an amount up to 10% by weight of the oral delivery agent, in some embodiments in an amount up to 5% by weight of the oral delivery agent, in some embodiments in an amount up to 2.5% by weight of the oral delivery agent, in some embodiments in an amount up to 1.0% by weight of the oral delivery agent, in some embodiments in an amount up to 0.75% by weight of the oral delivery agent, in some embodiments in an amount up to 0.5% by weight of the oral delivery agent, in some embodiments in an amount up to 0.25% by weight of the oral delivery agent, in some embodiments in an amount up to 0.1% by weight of the oral delivery agent, in some embodiments in an amount up to 0.05% by weight of the oral delivery agent, in some embodiments in an amount up to 0.01% by weight of the oral delivery agent, and in some embodiments in an amount less than 0.01% by weight of the oral delivery agent. In some embodiments, the amount of the low flavor antimicrobial liquid smoke derivative present in the oral delivery agent is measured as a dilution of the low flavor antimicrobial liquid smoke derivative in the oral delivery agent, particularly with respect to oral delivery agents that are liquids or pastes. Thus, the amount of the low flavor antimicrobial liquid smoke derivative can be present in the oral delivery agent in a dilution including, but not limited to a 1:2 dilution, a 1:4 dilution, a 1:8 dilution, a 1:16 dilution, a 1:20 dilution, a 1:32 dilution, a 1:40 dilution, a 1:64 dilution, a 1:80 dilution, a 1:128 dilution, a 1:160 dilution, a 1:256 dilution, a 1:320 dilution, a 1:512 dilution, a 1:640 dilution, a 1:1024 dilution, a 1:1280 dilution, a 1:2048 dilution, a 1:2560 dilution, a 1:4096 dilution, a 1:5120 dilution, a 1:8192 dilution, or a 1:10240 dilution.

The oral composition can also include additional breath freshening or oral health ingredients, which can themselves be anti-microbial ingredients. Exemplary additives include those flavor agents that are generally recognized as safe (GRAS) by the Food and Drug Administration of the United States of America. Further, the additional breath freshening or oral health ingredients can comprise food acceptable salts of zinc or copper, cooling agents, pyrophosphates or polyphosphates, and the like.

III.A.1. Gums

In some embodiments of the presently disclosed subject matter, the oral composition is provided in the form of a chewing gum. In some embodiments of the presently disclosed subject matter, the amount of the low flavor antimicrobial liquid smoke derivative is about 5% of the weight of the chewing gum product. In some embodiments of the presently disclosed subject matter, the amount of the low flavor antimicrobial liquid smoke derivative is about 2.5% of the weight of the chewing gum product. In some embodiments of the presently disclosed subject matter, the amount of the low flavor antimicrobial liquid smoke derivative is about 1% of the weight of the chewing gum product. In some embodiments of the presently disclosed subject matter, the amount of the low flavor antimicrobial liquid smoke derivative is about 0.5% of the weight of the chewing gum product. In some embodiments of the presently disclosed subject matter, the amount of the low flavor antimicrobial liquid smoke derivative is about 0.25% of the weight of the chewing gum product. In some embodiments of the presently disclosed subject matter, the amount of the low flavor antimicrobial liquid smoke derivative is about 0.1% of the weight of the chewing gum product. Accordingly, in some embodiments an effective amount of the low flavor antimicrobial liquid smoke derivative can range between about 0.1% by weight to about 5% by weight of the composition.

In general, a chewing gum composition typically comprises a water-soluble bulk portion, a water-insoluble chewable gum base portion and typically water-soluble flavoring agents. The water-soluble bulk portion dissipates with a portion of the flavoring agent over a period of time during chewing. The gum base portion is retained in the mouth throughout the chew.

The insoluble gum base generally comprises elastomers, resins, fats and oils, softeners and inorganic fillers. The gum base can or can not include wax. The insoluble gum base can constitute approximately 5% to about 95% by weight of the chewing gum, more commonly the gum base comprises 10% to about 50% of the gum, and in some embodiments approximately 25% to about 35% by weight, of the chewing gum.

In some embodiments, the chewing gum base of the presently disclosed subject matter contains about 20% to about 60% by weight synthetic elastomer, up to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Synthetic elastomers can include, but are not limited to, polyisobutylene with GPC weight average molecular weight of about 10,000 to about 95,000, isobutylene-isoprene copolymer (butyl elastomer), styrenecopolymers having styrene-butadiene ratios of about 1:3 to about 3:1, polyvinyl acetate having GPC weight average molecular weight of about 2,000 to about 90,000, polyisoprene, polyethylene, vinyl acetate vinyl laurate copolymer having vinyl laurate content of about 5% to about 50% by weight of the copolymer, and combinations thereof.

Exemplary, non-limiting ranges for polyisobutylene are 50,000 to 80,000 GPC weight average molecular weight; for styrene are 1:1 to 1:3 bound styrene; for polyvinyl acetate are 10,000 to 65,000 GPC weight average molecular weight, with the higher molecular weight polyvinyl acetates typically used in bubble gum base; and for vinyl acetate laurate, a vinyl laurate content of 10.

Natural elastomers can include natural rubber, such as smoked or liquid latex and guayule, as well as natural gums, such as jelutong, lechi caspi, perillo, sorva, massaranduba balata, massaranduba chocolate, nispero, rosindinha, chicle, gutta hang kang, and combinations thereof. The synthetic elastomer and natural elastomer concentrations can vary depending on whether the chewing gum in which the base is used is adhesive or conventional, bubble gum or regular gum, as discussed below. Exemplary natural elastomers include jelutong, chicle, sorva and massaranduba balata.

Elastomer plasticizers can include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha, beta, and/or any suitable combinations of the foregoing. The elastomer plasticizers can also vary depending on the specific application, and on the type of elastomer which is used.

Fillers/texturizers can include magnesium and calcium carbonate, ground limestone, silicate types such as magnesium and aluminum silicate, clay, alumina, talc, titanium oxide, mono-, di- and tri-phosphate, cellulose polymers, such as wood, and combinations thereof.

Softeners/emulsifiers can include tallow, hydrogenated tallow, hydrogenated and partially hydrogenated vegetable oils, cocoa butter, glycerol monostearate, glycerol triacetate, lecithin, mono and triglycerides, acetylated monoglycerides, fatty acids (for example stearic, pamitic, oleic and linoleic acids), and combinations thereof.

Colorants and whiteners can include FD&C dyes and lakes, fruit and vegetable extracts, titanium dioxide, and combinations thereof.

The base can also include a wax. An example of a wax-free gum base is disclosed in U.S. Pat. No. 5,286,500, the disclosure of which is incorporated herein by reference.

In addition to a water insoluble gum base portion, a typical chewing gum composition includes a water soluble bulk portion and one or more flavoring agents. The water soluble portion can include bulk sweeteners, high intensity sweeteners, flavoring agents, softeners, emulsifiers, colors, acidulants, fillers, antioxidants, and other components that provide desired attributes.

Softeners can be added to the chewing gum in order to optimize the chewability and mouthfeel of the gum. The softeners, which are also known as plasticizers and plasticizing agents, generally constitute between approximately 0.5% to about 15% by weight of the chewing gum. The softeners can include glycerin, lecithin, and combinations thereof. Aqueous sweetener solutions such as those containing sorbitol, hydrogenated starch hydrolysates, corn syrup and combinations thereof, can also be used as softeners and binding agents in chewing gum.

Bulk sweeteners, or bulking agents, include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide components commonly known in the chewing gum art, including but not limited to, sucrose, dextrose, maltose, dextrin, dried invert sugar, fructose, levulose, galactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High intensity artificial sweeteners can also be used, alone or in combination, with the above. Exemplary sweeteners include, but are not limited to, sucralose, aspartame, NAPM derivatives such as neotame, salts of acesulfame, altitame, saccharin and its salts, cyclamic acid and its salts, glycyrrhizinate, dihydrochalcones, thaumatin, monellin, and the like, alone or in combination. In order to provide longer lasting sweetness and flavor perception, it can be desirable to encapsulate or otherwise control the release of at least a portion of the artificial sweetener. Such techniques as wet granulation, wax granulation, spray drying, spray chilling, fluid bed coating, coacervation, and fiber extension can be used to achieve the desired release characteristics.

Combinations of sugar and/or sugarless sweeteners can be used in chewing gum. Additionally, the softener can also provide additional sweetness such as with aqueous sugar or alditol solutions.

If a low calorie gum is desired, a low caloric bulking agent can be used. Examples of low caloric bulking agents include: polydextrose; raftilose, raftilin; fructooligosaccharides (NutraFlora); Palatinose oligosaccharide; guar gum hydrolysate (Sun Fiber); or indigestible dextrin (Fibersol). However, other low calorie bulking agents can be used.

A variety of flavoring agents can also be used, if desired. The flavor can be used in amounts of about 0.1 to about 15 weight percent of the gum, and in some embodiments, about 0.2% to about 5% by weight. Flavoring agents can include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components can also be used. Natural and artificial flavoring agents can be combined in any sensorially acceptable fashion. Flavoring can include a cooling agent to enhance the flavor and perceived breath freshening of the product. Cooling agents include menthol, ethyl p-menthane carboxamide, N,2,3-trimethyl-2-isopropyl-butanamide, menthyl glutarate (Flavor Extract Manufacturing Association (FEMA 4006)), menthyl succinate, menthol PG carbonate, menthol EG carbonate, menthyl lactate, menthone glyceryl ketal, menthol glyceryl ether, N-tertbutyl-p-menthane-3-carboxamide, p-menthane-3-carboxylic acid glycerol ester, methyl-2-isopryl-bicyclo(2.2.1), heptane-2-carboxamide, menthol methyl ether and combinations thereof.

In addition to the low flavor antimicrobial liquid smoke derivative of the presently disclosed subject matter, additional active ingredients or medicaments can be added for various purposes. If the medicament or active is water soluble in the chewing gum, it can include a base/emulsifier system which leads to the desired concentration of the medicament in the saliva (more hydrophilic balance). If the medicament or active is water insoluble, the chewing gum can include a base/emulsifier system which leads to the desired concentration of the medicament in the saliva (more lipophilic balance).

In manufacturing the chewing gum including the active agent or ingredient, the active agent or medicament is added, in some embodiments, early on in the mix. The smaller the amount of active ingredient used, the more necessary it becomes to preblend that particular ingredient to assume uniform distribution throughout the batch of gum. Whether a preblend is used or not, the active agent or medicament should be added within the first five minutes of mixing. For faster release, the active agent can be added late in the process.

Optionally, the chewing gum of the presently disclosed subject matter can include additional breath freshening, anti microbial, or oral health ingredients, such as food acceptable metallic salts selected from zinc and copper salts of gluconic acid, zinc and copper salts of lactic acid, zinc and copper salts of acetic acid, zinc and copper salts of citric acid, copper chlorophyll and combinations thereof. Further, anti-microbial essential oils and flavor components such as peppermint, methyl salicylate, thymol, eucalyptol, cinnamic aldehyde, polyphosphate, pyrophosphate and combinations thereof can be added to the gum composition. Dental health ingredients, such as fluoride salts, phosphate salts, proteolytic enzymes, lipids, anti-microbials, calcium, electrolytes, protein additives, dental abrasives and combinations thereof can also be added to the gum composition.

In general, chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned.

Generally, the ingredients are mixed by first melting the gum base and adding it to the running mixer. The base can also be melted in the mixer itself. Color or emulsifiers can also be added at this time. A softener such as glycerin can also be added at this time, along with syrup and a portion of the bulking agent. Further parts of the bulking agent are added to the mixer. Flavoring agents are typically added with the final portion of the bulking agent. Other optional ingredients are added to the batch in a typical fashion, well known to those of ordinary skill in the art.

The entire mixing procedure typically takes from five to fifteen minutes, but longer mixing times can sometimes be required. Those skilled in the art will recognize that many variations of the above described procedure can be followed.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacture, on the one hand, involves the dispersive (often high shear) mixing of difficult-to-blend ingredients such as elastomer, filler, elastomer plasticizer, base softeners/emulsifiers and sometimes wax, and typically requires long mixing times. Chewing gum product manufacture, on the other hand, involves combining the gum base with more delicate ingredients such as product softeners, bulk sweeteners, high intensity sweeteners and flavoring agents using distributive (generally lower shear) mixing, for shorter periods.

The production of chewing gums as oral care products is described in U.S. Pat. No. 6,905,673, the contents of which, including all references cited therein, is incorporated by reference herein in its entirety.

III.A.2. Edible Films

In some embodiments, an effective amount of a the low flavor antimicrobial liquid smoke derivative of the presently disclosed subject matter, such as described above, is present in an edible film formulation. In some embodiments, the low flavor antimicrobial liquid smoke derivative is present in an amount up to 10% by weight of the edible film formulation. In some embodiments, the amount of the low flavor antimicrobial liquid smoke derivative is about 8% of the weight of the edible film product. In some embodiments, the low flavor antimicrobial liquid smoke derivative is present in the amount of about 5% by weight of the edible film product. Considering the potency of the low flavor antimicrobial liquid smoke derivative as described in the in vitro studies disclosed herein, about 1% by weight or less of the low flavor antimicrobial liquid smoke derivative can also be effective in bactericidal properties. Accordingly, an effective amount of the low flavor antimicrobial liquid smoke derivative can range between about 0.1% by weight to about 10% by weight of the edible film composition.

The presently disclosed subject matter provides in some embodiments edible film formulations for oral care and/or treatment and methods of using and making same. In some embodiments, the edible films of the presently disclosed subject matter comprise pullulan and are prepared by methods that are as set forth, for example, in U.S. Pat. Nos. 5,089,307 or 5,620,757, the disclosure of each of which is incorporated by reference in its entirety.

Additional methods can include the use of film forming agents other than pullulan. For example, methods for producing edible films comprising mixtures of at least three types of film forming agents, such as maltodextrins, fillers (for example, microcrystalline cellulose (MCC)) and hydrocolloids (for example, sodium aliginate), are provided in PCT International Patent Application Publication No. WO 2007/011504, the disclosure of which is incorporated by reference in its entirety.

The edible films of the presently disclosed subject matter can be utilized to deliver or release additional oral care agent(s) besides the low flavor antimicrobial liquid smoke derivative. Such agents include anti-microbial agents and salivary stimulants to treat, for example, halitosis, dental plaque, gingivitis, xerostomia, dry mouth, like oral conditions or combinations thereof. Further, the oral care edible film can act as a breath freshener effective against malodor.

The oral cleansing and breath freshening effects of the edible film of the presently disclosed subject matter can be achieved by entrapping the oral care agents within the oral cavity to provide extended efficacy. In this regard, the highly dissolvable edible film can act as a medium through which a pharmaceutically active oral agent can be administered via a mucous membrane of the oral cavity.

Further, the edible films can include a variety of other suitable ingredients, such as softeners, colorants, flavoring agents, emulsifiers, surface active agents, thickening agents, binding agents, sweeteners, fragrances, other like ingredients or combinations thereof.

Any suitable food-grade bulk filler can also be added to the edible film. This can reduce any slimy texture as well as provide structure to the film thereby making it more palatable. In an embodiment, the filler can constitute about 5% to about 30% by dry weight of the film, preferably about 15% to about 25% by dry weight. The filler can include, for example, microcrystalline cellulose, cellulose polymers, such as wood, magnesium and calcium carbonate, ground limestone, silicates, such as magnesium and aluminum silicate, clay, talc, titanium dioxide, mono-calcium phosphate, di-calcium phosphate, tri-calcium phosphate, other like bulk fillers or combinations thereof.

A variety of other suitable ingredients can be added to the edible film of the presently disclosed subject matter. For example, any suitable medicament for oral cleansing, breath freshening or the like can be added to the film formulation. The medicaments can include, for example, a pH control agent, such as urea and buffers, inorganic components for tartar or caries control, such as phosphates and fluorides, a breath freshening agent such as zinc gluconate, an anti-plaque/anti-gingivitis agent, such as cholorhexidene, CPC, and triclosan, a saliva stimulating agent including, for example, food acids such as citric, lactic, maleic, succinic, ascorbic, adipic, fumaric and tartaric acids, a pharmaceutical agent, a nutraceutical agent, a vitamin, a mineral, other like medicaments or combinations thereof.

The edible film formulations of the presently disclosed subject matter can also include colorants or coloring agents which can be used in any suitable amount to produce the desired color. Coloring agents can include, for example, natural food colors and dyes suitable for food, drug and cosmetic applications. The colorants are typically knows as FD&C dyes and lakes.

A variety of flavoring agents can also be added to the edible films. Any suitable amount and type of artificial and/or natural flavoring agents can be used in any sensorially acceptable fashion. For example, the flavor can constitute about 0.1% to about 20% by dry weight of the film, preferably about 10% to 15%. The flavoring agent can include, for example, essential oils, synthetic flavors or mixtures including but not limited to oils delivered from plants and fruits such as citrus oils, fruit essences, peppermint oil, spearmint oil, other mint oils, clove oils, oil of wintergreen, anise and the like, flavor oils with germ killing properties such as menthol, eucalyptol, thymol, like flavoring agents or combinations thereof.

The flavor can be enhanced and evenly distributed throughout the product by emulsification. Any suitable amount and type of natural and/or synthetic food grade emulsifier can be used. For example, the emulsifier can include lecithin, food-grade non-ionic emulsifiers, such as fatty acids (C₁₀-C₁₈), mono and diacyl glycerides, ox bile extract, polyglycerol esters, polyethylene sorbitan esters, propolyene glycol, sorbitan monopalmitate, sorbitan monosterate, sorbitan tristearate, enzyme modified lecithin, hyroxylated lecithins, other like emulsifiers or combinations thereof.

The flavors can be emulsified by any suitable emulsification process, such as mechanical processing, vigorous stirring, intense pressure fluctuations that occur in turbulent flow such as homogenization, sonication, colloid milling and the like.

III.A.3. Confectioneries

In some embodiments, an effective amount for anti-microbial benefit of the low flavor antimicrobial liquid smoke derivative of the presently disclosed subject matter, such as described above, is provided in a confectionery formulation. Confectionery products that comprise the low flavor antimicrobial liquid smoke derivative can be, for example, hard candies, chewy candies, coated chewy center candies, ore tabletted candies. By way of example, a hard candy is primarily comprised of corn syrup and sugar, and derives its name from the fact that it contains only 1.0% and 4% moisture. In appearance, these types of candies are solid, but they are actually supercooled liquids, which are far below their melting points. There are different types of hard candies. Glass types are usually clear or made opaque with dyes; and Grained types, which are always opaque.

The continuous process of making the deposited glass types with a sugar base are as follows. Corn syrup is spread over a cylinder heated by high pressure steam. Rapid heat exchange causes the water in the syrup to evaporate. The cooked syrup is discharged, colors and flavors are added. The syrup is cooled and deposited onto a stainless steel conveyor. The syrup can be conveyed directly to hoppers which then discharge directly into molds.

The candy is conveyed to batch rollers, which shapes and sizes the batch. The candy enters a former, which shapes the individual pieces into discs, balls, barrels, etc. The present invention can be made into any shape, circles, squares, triangles etc., also into animal shapes or any other novelty molding available. The candy is then cooled, wrapped and packaged.

For grained types of candy, water and sugar are the basic components being mixed with other ingredients, and cooked at high temperatures (143-155° C.; i.e., approximately 290-310° F.), causing the water to turn to steam. The product is transferred to a cooling wheel, where it is collected in about 68 kg (approximately 150 pound) batches, placed in a pulling machine to aerate the product, and the flavor is added.

The candy is transferred to batch rollers where it is shaped and sized. The candy then enters a former, which shapes the individual pieces. The candy is cooled at a relative humidity of 35% and enters a rotating drum where it is coated with a fine sugar. The candy is then conveyed to the graining room for four hours at approximately 32° C. (90° F.) and 60% humidity. The entrapped air and moisture causes the product to grain.

III.A.4. Other Formulations

The low flavor antimicrobial liquid smoke derivatives of the presently disclosed subject matter can also be provided in the form of a dentifrice, a lozenge, a mouthwash, and/or a mouth spray. Methods for embedding these formulations with low flavor antimicrobial liquid smoke derivatives of the presently disclosed subject matter are similar to those described hereinabove with respect to the other oral delivery agents described. Methods for preparing dentifrices, lozenges, mouthwashes, and mouth sprays are known, and include those methods set forth in U.S. Pat. Nos. 4,994,441; 5,688,495; 6,455,031; 6,509,007; 6,936,589; and 6,974,859; the disclosure of each of which is incorporated by reference in its entirety.

III.B. Methods

The presently disclosed subject matter also provides methods for reducing the number and/or activity of bacteria in the oral cavity of a consumer comprising the steps of providing an oral composition comprising a low flavor antimicrobial liquid smoke derivative in an amount sufficient to kill or deactivate oral bacteria, whereby the bacteria in the oral cavity of the consumer is reduced or inactivated by the treatment.

In some embodiments, the presently disclosed subject matter provides methods for oral cleansing. In some embodiments, the methods comprise applying an oral composition to the oral cavity of a subject, wherein the oral composition comprises (a) an oral delivery agent; and (b) an effective amount of a low flavor antimicrobial liquid smoke derivative. As used herein, the phrase “oral delivery device” refers to a formulation to which a low flavor antimicrobial liquid smoke derivative can be added to deliver the low flavor antimicrobial liquid smoke derivative to the oral cavity of a subject. Exemplary oral delivery devices include, but are not limited to chewing gums, edible films, confectionaries, dentifrices, lozenges, mouthwashes, and mouth sprays.

The presently disclosed subject matter also provides methods for inhibiting growth of a microorganism in the oral cavity of a subject. As used herein, the term “inhibiting” refers to causing a measurable decrease in the number, viability, and/or growth rate of a microorganism present within the subject's oral cavity. Thus, “inhibiting” is understood to include not just an absolute bar to viability and/or growth, but also a reduction in any of these parameters. In some embodiments, the methods comprise applying an oral composition comprising an oral care delivery agent and an antimicrobial agent comprising an effective amount of a low flavor antimicrobial liquid smoke derivative to the oral cavity of the subject for a time sufficient to inhibit growth of a microorganism in the oral cavity of the subject.

### EXAMPLES

The following Examples have been included to illustrate modes of the presently disclosed subject matter. Certain aspects of the following Examples are described in terms of techniques and procedures found or contemplated by the present inventors to work well in the practice of the presently disclosed subject matter. These Examples illustrate standard practices of the inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Production of Representative Natural Low-Flavor Anti-Microbial Liquid Smoke Derivatives

Three different representative natural low-flavor anti-microbial liquid smoke derivatives were produced using standard techniques. Agent No. 1 was derived from pyrolysis of lignin-free hardwood pellets using a conventional liquid smoke making process. This process included the following basic steps: (1) smoke was generated by pyrolysis of a pre-dried raw material; (2) scrubbing the smoke into a water phase; (3) settling and removal of tars; (4) further purification to remove strong smoke flavor components and/or concentration; e.g. filtering, carbon absorption, and/or evaporation; (5) standardizing and stabilizing to meet specifications.

Agent No. 2 was a spray-dried version of Agent No. 1, and was produced by the following procedure: (1) adding a natural polysaccharide, for example potato maltodextrin, to Agent No. 1 to adjust the final solid level to 35%-40%; (2) homogenization; (3) spray drying with inlet and outlet temperatures at 350° F. and 215° F., respectively.

Agent No. 3 was a concentrated version of Agent No. 1 that was obtained by removing moisture with vacuum evaporation.

Agent Nos. 1-3 had the properties set forth in Table 2.

TABLE 2
Properties of Representative Liquid Smoke Derivatives

| Characteristic | Agent No. 1 | Agent No. 2 | Agent No. 3 |
|---|---|---|---|
| % Acidity | 0.8-3.5 | 0.2-2.5 | 0.8-4.5 |
| pH | 3.5-5.5 | n.a. | 2.2-5.5 |
| Specific Gravity | 1.055 to 1.175 | n.a. | 1.065-1.195 |
| Carbonyl | 0.5-8.0 g/100 ml | 0.5-8 g/100 g | 3-25 g/100 ml |
| Phenol | 0.0-1.2 mg/ml | 0.0-0.7 mg/g | 0.0-2.4 mg/ml |
| Moisture | n.a. | <6% | n.a. |

### Example 2

### Anti-Microbial Activities of Representative Natural Low-Flavor Anti-Microbial Liquid Smoke Derivatives

Agent Nos. 1-3 were tested for their ability to inhibit the growth of various strains of bacterial that are known to be relevant to tooth decay and/or periodontal disease. These bacterial strains included Streptococcus mutans (dental caries); Porphorymonas gingivalis (periodontal disease); and Fusobacterium nucleatuin ssp. polymorphum (periodontal disease). In various experiments, the minimum concentration of an Agent that inhibits the growth of the target organism (MIB) and/or the minimum concentration of an Agent that kills the target organism (MIC) were determined.

In a first set of experiments, the minimal inhibitory concentration (MIC) of each of the three Agents against each of three oral microflora was tested. Bacteria were cultured in the presence of successive two-fold dilutions of each Agent until the most dilute concentration of the Agent that inhibited bacterial growth was determined. This was defined to be the MIC or minimal inhibitory concentration.

More particularly, the minimal inhibitory concentration (MIC) of each of the three Agents against each of three oral microflora was determined. The MIC was defined as the lowest concentration of an antimicrobial agent that prevented visible growth of a microorganism in an agar or broth dilution susceptibility test. The tests were performed according to the procedures set forth in the National Committee for Clinical Laboratory Standards (NCCLS; now called the Clinical and Laboratory Standards Institute (CLSI)) publication Methods for Antimicrobial Susceptibility Testing of Anaerobic Bacteria; Approved Standard—Sixth Edition, Volume 24 No. 2, NCCLS document No. M11-A6 (ISBN 1-56238-517-8), NCCLS, Wayne, Pa., United States of America.

Dilutions of Antimicrobial Agents. Dilutions of each Agent were prepared and tested for anti-microbial activity. For Agent No. 2, which is a spray-dried version of Agent No. 1, 20 ml (20 g) of sterile, distilled water was first added to 10 g of Agent No. 2 before the dilution series were prepared (i.e., the amount of Agent No. 2 in the stock solution corresponded to an initial 1:3 weight:weight dilution). A series of dilutions were prepared for each Agent by diluting the Agent with sterile, distilled water by making successive 1:2, 1:4, 1:8, etc. dilutions of the stocks by using the dilution format described in Table 4 of the NCCLS reference cited supra. These dilutions represented the intermediate (10×) solutions (i.e., 2 ml of these solutions were added to the 18 ml of agar just before pouring the plates.

Preparation of Agar Dilution Plates. A total of 20 ml of medium was used for each Petri dish. The medium for the growth of the three bacterial strains was Brucella Agar supplemented with hemin (5 μg/ml), vitamin K (1 μg/ml), and 5% laked sheep blood. The medium was prepared without blood and refrigerated in tubes (17 ml/50 ml tube) until just prior to testing. The base medium was then melted, cooled to 48 to 50° C., and 1 ml of sterile laked sheep blood was added (the final ratio, after adding the diluted Agent, was 1 ml of blood per 20 ml medium. Just before pouring the plates, one part of a 10× solution (i.e., 10× the desired final concentration) of antimicrobial agent was added to nine parts agar. So, for each plate, 2 ml of antimicrobial agent (10×) was added to the 18 ml of Brucella agar-blood mixture maintained at 48 to 50° C. in tubes. For control plates, 2 ml of sterile, distilled water was used in place of 2 ml of diluted agents. The tubes were then gently inverted several times to mix ingredients and the agar solution poured into Petri plates. After solidification, the plates were placed in an incubator at 37° C. for 0.5 hour with their lids ajar to remove excess moisture. Plates were used within 2 hours of preparation.

Inoculum Preparation for Dilution Plates. The test organisms were grown on supplemented Brucella blood agar plates (described below) with 5% defibrinated sheep blood (Colorado Serum Co., Denver, Colo., United States of America) for 24-48 hours prior to carrying out the experiments.

Turbidity Standard for Inoculum Preparation. To standardize the inoculum density for susceptibility testing, a BaSO₄ turbidity standard equivalent to a 0.5 McFarland standard was used. Colonies of bacteria from supplemented Brucella blood agar plates incubated for 24-48 hours were suspended in BHI broth (Difco Laboratories, Detroit, Mich., United States of America) and the turbidity adjusted to that of the McFarland standard.

Inoculation of Agar Dilution Plates. Aliquots of 2 microliters (2 μl) of the standardized bacterial suspensions were applied in triplicate onto the agar surface of plates containing from zero (0) to up to a 1/10,240th dilution of one of Agent Nos. 1-3 (see Tables 3-5). The plates were incubated anaerobically from 42-48 hours as 37° C.

Anaerobic Incubation. Plates were incubated in an anaerobic chamber (DW Scientific, Shipley, West Yorkshire, England) for 42-48 hr at 37° C. At this time, good growth of all three species was observed on the control plates. An indicator to confirm anaerobiosis was included.

Determination of the MIC. After incubation of the plates with diluted agents for 42-48 hours, they were examined for presence or absence of growth by comparison with growth of the bacteria on the control plates. In cases where there was questionable growth, the MICs were determined by comparing the test plates with the examples of results of experiments shown in the NCCLS reference (see NCCLS Reference, FIG. 2: MIC Endpoints—Agar Dilution).

TABLE 3
Growth Inhibition of Streptococcus mutans

| | Concentration* | | Agent No. | |
|---|---|---|---|---|
| | (Dilutions of stocks) | Agent No. 1 | 2* | Agent No. 3 |
| Step 1 | 1/20^{th} | NG | NG | NG |
| Step 2 | 1/40^{th} | NG | NG | NG |
| Step 3 | 1/80^{th} | NG | NG | NG |
| Step 4 | 1/160^{th} | NG | NG | NG |
| Step 5 | 1/320^{th} | NG | GR | NG |
| Step 6 | 1/640^{th} | GR | GR | NG |
| Step 7 | 1/1280^{th} | GR | GR | GR |
| Control | H₂0 (no Agent) | GR | GR | GR |

*The Agent No. 2 concentrations are 3-fold more diluted than the dilutions of stock listed in the Table since Agent No. 2 was originally diluted 1:3 (w/w) in sterile, distilled water prior to the stocks being diluted. Thus, for Agent No. 2 the 1/20^{th} dilution is effectively a 1/60^{th} dilution, the 1/160^{th} dilution is effectively a 1/480^{th} dilution, etc.

TABLE 4
Growth Inhibition of Fusobacterium nucleatum

| | Concentration* | | | |
|---|---|---|---|---|
| | (Dilutions of stocks) | Agent No. 1 | Agent No. 2 | Agent No. 3 |
| Step 1 | 1/20^{th} | NG | NG | NG |
| Step 2 | 1/40^{th} | NG | NG | NG |
| Step 3 | 1/80^{th} | NG | NG | NG |
| Step 4 | 1/160^{th} | NG | NG | NG |
| Step 5 | 1/320^{th} | NG | NG | NG |
| Step 6 | 1/640^{th} | NG | NG | NG |
| Step 7 | 1/1280^{th} | NG | NG | NG |
| Step 8 | 1/2560^{th} | NG | NG | NG |
| Step 9 | 1/5120^{th} | GR | GR | NG |
| Step 10 | 1/10,240^{th} | GR | GR | GR |
| Control | H₂0 (no agent) | GR | GR | GR |

TABLE 5
Growth Inhibition of Porphrymonas gingivalis

| | Concentration* | | | |
|---|---|---|---|---|
| | (Dilutions of stocks) | Agent No. 1 | Agent No. 2 | Agent No. 3 |
| Step 1 | 1/20^{th} | NG | NG | NG |
| Step 2 | 1/40^{th} | NG | NG | NG |
| Step 3 | 1/80^{th} | NG | NG | NG |
| Step 4 | 1/160^{th} | NG | NG | NG |
| Step 5 | 1/320^{th} | NG | NG | NG |
| Step 6 | 1/640^{th} | NG | NG | NG |
| Step 7 | 1/1280^{th} | GR | GR | NG |
| Step 8 | 1/2560^{th} | GR | GR | NG |
| Step 9 | 1/5120^{th} | GR | GR | NG |
| Control | H₂0 (no agent) | GR | GR | GR |

Legend for Tables 3-5
* Shows the dilution of the antimicrobial agents to which the bacteria were exposed in the agar dilution plates (see Column 2, below).
Column 1—Steps 1 through 7. These just refer to the dilution “steps”—each step being a twofold dilution of the previous sample that was used.
Column 2—1/20th thru 111280th. These refer to the precise concentrations (dilutions) of the samples received from MT that were present in the plates on which the bacteria were grown. In step 1, for example, a 1/2 dilution of the stock had been previously prepared and 2 ml of it were added to the 18 ml of media before pouring the plates (see Materials and Methods).
NG=No Growth of bacteria
GR=Growth of bacteria
NG=the dilution of sample which indicates the MIC, minimal inhibitory concentration (dilution).
Control—Plates prepared with 2 ml of sterile water instead of an antimicrobial product.

### Discussion of Example 2

All three agents exhibit potent antimicrobial activity against the three bacterial species tested. The effectiveness of each agent, relative to the other two, was determined by identifying the most dilute concentration (dilution; MIC) that inhibited the growth of a microbe compared to the MICs of the other two agents. This was done for each bacterial species. The more dilute the concentration of the agent that inhibited growth, the more potent it was deemed to be.

In Table 3, for example, a 1/640th dilution of Agent No. 3 represented the MIC for S. mutans, whereas a 1/320th dilution of Agent No. 1 and a 1/160th dilution of Agent No. 2 represented their MICs. Thus, Agent No. 3 had twice the antimicrobial activity of Agent No. 1 and four times the antimicrobial activity of Agent No. 2 against S. mutans.

Similarly, with F. nucleatum (see Table 4) a 1/5120th dilution of Agent No. 3 represented the MIC, whereas a 1/2560th dilution of the other two Agents represented their MICs. Thus, Agent No. 3 had twice the antimicrobial activity of Agent Nos. 1 and 2 against F. nucleatum.

With P. gingivalis (see Table 5), Agent No. 3 (1/2560th dilution) is four times as effective as the other two Agents (MICs=1/640th dilution).

To summarize:
Comparative Effectiveness of Each Agent Against Each Species
   against S. mutans Agent No. 3 2×>Agent No. 1 2×>Agent No. 2
   against F. nucleatum Agent No. 3 2×>Agent Nos. 1 and 2
   against P. gingivalis Agent No. 3 4×>Agent Nos. 1 and 2
Comparative Sensivity of Each Species Against Each Agent
   F. nucleatum is most sensitive to each Agent.
   P. gingivalis is next most sensitive to each Agent.
   S. mutans is most resistant to each Agent.
Overall Effective of Each Agent
   Agent No. 3 is the most potent agent against all 3 species.
   Agent No. 1 is equivalent to Agent No. 2 against F. nucleatum and P. gingivalis, and twice as effective as Agent No. 2 against S. mutans.
   Agent No. 2 is the less effective against S. mutans than Agent Nos. 1 and 2, but is equivalent to Agent No. 1 against F. nucleatum and P. gingivalis.

### Example 3

### MIC/MIB Values of Agent Nos. 1 and 2 Compared to a Commercial Oral Care Product

The MIC/MIB values of various dilutions of Agent Nos. 1 and 2 against S. mutans, F. nucleatum ssp. polymorphm, and/or P. gingivalis were determined and compared, in certain instances, to MIC/MIB values with COOLMINT® LISTERINE® mouthwash (Pfizer, Morris Plains, N.J., United States of America) as a control. Three different independent laboratories performed the comparisons, and the results are summarized in Tables 6-8.

TABLE 6
MIC and MIB Values - Laboratory A

| | MIC | | | MIB | | |
|---|---|---|---|---|---|---|
| | Agent | Agent | | Agent | Agent | |
| | No. 1 | No. 2 | Control | No. 1 | No. 2 | Control |
| S. mutans | 0.4%* | 0.1% | >25% | 0.8%** | 0.4% | >25% |
| F. nucleatum | 0.1% | 0.1% | 16% | 0.1% | 0.1% | 16% |

*Minimum % concentration of the agent at which inhibited the growth of the target organism;
**Minimum % concentration of the agent at which killed the target organism.

TABLE 7
MIC and MIB Values - Laboratory B

| | MIC | | | MIB | | |
|---|---|---|---|---|---|---|
| | Agent | Agent | | Agent | Agent | |
| | No. 1 | No. 2 | Control | No. 1 | No. 2 | Control |
| S. mutans | 1.56% | 0.05% | >12.5% | 1.56% | 0.05% | >12.5% |

TABLE 8
MIC and MIB Values - Laboratory C

| | MIC | | |
|---|---|---|---|
| | Agent | Agent | Agent |
| | No. 1 | No. 2 | No. 3 |
| S. mutans | 0.63% | 0.31% | 0.16% |
| F. nucleatum | 0.04% | 0.04% | 0.02% |
| P. gingivalis | 0.16% | 0.16% | 0.04% |

### Discussion of Example 3

All three agents demonstrated strong in vitro efficacy at inhibiting/killing S. mutans, the most common cariogenic bacterium in human mouth. Agent No. 2 was consistently shown stronger than Agent No. 1, the liquid form from which Agent No. 2 was spray dried. The testing that corresponded to the results presented in Table 8 also demonstrated that all Agents were highly effective against F. nucleatum and P. gingivalis, which are known to be involved in periodontal disease and halitosis in humans.

It should be noted that the absolute differences in the results among the various laboratories were presumably due to variations among the actual protocols used by the individual labs.

### Example 4

### Taste Testing of a Chewing Gum Containing Agent No. 2

Agent No. 2 was mixed into chewing gum formulations at levels ranging from 0.01% to 2.5% (w/w). Due to the unique low flavor character of the Agent, at the 1.5% usage level no objectionable flavor was tasted in the application.

The results from a representative sample formulation (0.25% level) is presented in Table 9 below.

TABLE 9
Composition of a Chewing Gum Containing Agent No. 2

| Component | % |
|---|---|
| Gum base | 30 |
| Sorbitol powder | 47.5 |
| Mannitol powder | 8 |
| Lycasil | 8 |
| Glycerin | 5 |
| Lecytin | 0.5 |
| Aspartame | 1 |
| TOTAL | 100 |

0.25% orange flavor was added to the above gum to provide flavor, and testers remarked that the gum had a typical fresh orange flavor. 0.25% Agent No. 2 was then also added (i.e., to the above gum plus 0.25% orange flavor), and the testers noted no off-flavor due to addition of the Agent.

### Example 5

### Taste Testing of a Candy Containing Agent No. 2

A sugar free syrup filling for a chewy candy was also prepared. The base composition of the filling is set forth in Table 10.

TABLE 10
Composition of a Sugar-free Candy Filling
Containing Agent No. 2

| Component | % |
|---|---|
| Maltitol syrup | 95 |
| Glycerin | 5 |
| Acesulfame k | 0.1 |
| Citric acid | a.n. |
| Color | a.n. |
| TOTAL | 100 |

Agent No. 3 was mixed into the sugar free syrup filling in formulations at the range of 0.01% to 50% level. In one example, orange flavor was added to 0.025% in the filling, and in another example, mint flavor was added to 0.20% in the filling. In both examples, Agent No. 3 was added to 0.5% in the filling. In each case, testers reported no off-flavor perceived due to the addition of Agent No. 3.

### Example 6

### Tests of Antimicrobial Activities of Oral Care Products

Gums, liquid oral rinses (e.g., mouthwashes), and/or confections, are loaded with a low flavor antimicrobial liquid smoke derivative of the presently disclosed subject matter in amounts ranging from 0.1% to 50%. The loaded compositions are employed as would be appropriate for the type of composition, and different resident times in the oral cavity of a subject are employed (e.g., 1 minute, 5 minutes, 10 minutes, 15 minutes, 20 minutes, etc.). Cultures of oral flora are taken and the number of bacteria from various species (e.g., S. mutans, F. nucleatum ssp. polymorphm, and P. gingivalis) is compared before and after treatment with the composition.

### REFERENCES

The references listed below, as well as all references cited in the present specification, including but not limited to patents, patent application publications, and non-patent literature, are incorporated herein by reference to the extent that they supplement, explain, provide a background for, or teach methodology, techniques, and/or compositions employed herein.

NCCLS document No. M11-A6, Methods for Antimicrobial Susceptibility Testing of Anaerobic Bacteria; Approved Standard—Sixth Edition, Volume 24 No. 2, (ISBN 1-56238-517-8), NCCLS, Wayne, Pa., United States of America.

PCT International Patent Application Publication No. WO 2007/011.

U.S. Pat. Nos. 3,873,741; 4,250,199; 4,298,435; 4,431,032; 4,994,441; 5,043,174; 5,089,307; 5,286,500; 5,620,757; 5,637,339; 5,688,495; 6,214,395; 6,455,031; 6,509,007; 6,905,673; 6,936,589; 6,974,859.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. An oral composition for freshening the breath of a consumer of the oral composition, the oral composition comprising:
(a) an oral delivery agent; and
(b) an antimicrobial agent comprising an effective amount of a low flavor antimicrobial liquid smoke derivative.

2. The oral composition of claim 1, wherein the oral delivery agent is selected from the group consisting of a chewing gum, an edible film, a confectionary, a dentifrice, a lozenge, a mouthwash, and a mouth spray.

3. The oral composition of claim 1, wherein the low flavor antimicrobial liquid smoke derivative comprises:
(a) titratable acidity in a concentration of 0 to about 6% weight per unit volume (w/v);
(b) at least about 3% weight per unit volume (w/v) carbonyl; and
(c) phenolics in a concentration of less than about 2.5 mg/ml.

4. The oral composition of claim 3, wherein the low flavor antimicrobial liquid smoke derivative comprises carbonyl in a concentration of about 8.0 to about 12.0% weight per unit volume (w/v), and a pH of less than about 6.0.

5. The oral composition of claim 4, wherein the low flavor antimicrobial liquid smoke derivative comprises carbonyl in a concentration of about 3.0 to about 8.0% weight per unit volume (w/v), phenolics in a concentration of less than about 1 mg/ml, and a pH of about 2.0 to about 5.5.

6. The oral composition of claim 3, wherein the low flavor antimicrobial liquid smoke derivative comprises carbonyl of at least 10% weight per unit volume (w/v).

7. The oral composition of claim 1, wherein the low flavor antimicrobial liquid smoke derivative is produced by processing liquid smoke through an evaporator to separate and condense the low boiling elements thereof to produce the derivative of liquid smoke.

8. A chewing gum composition for freshening the breath of a consumer, the composition comprising:
(a) a water soluble bulk portion;
(b) at least one flavoring agent;
(c) a gum base portion; and
(d) an effective amount of a low flavor antimicrobial liquid smoke derivative.

9. A confectionary composition for freshening the breath of a consumer, the composition comprising:
(a) at least one sugar, sugar alcohol, sugar substitute, or a combination thereof; and
(b) an effective amount of a low flavor antimicrobial liquid smoke derivative.

10. An edible film composition comprising:
(a) an effective amount of a film forming agent; and
(b) an effective amount of a low flavor antimicrobial liquid smoke derivative.

11. A method for oral cleansing, the method comprising applying an oral composition to the oral cavity of a subject, wherein the oral composition comprises:
(a) an oral delivery agent; and
(b) an effective amount of a low flavor antimicrobial liquid smoke derivative.

12. The method of claim 11, wherein the low flavor antimicrobial liquid smoke derivative comprises:
(a) titratable acidity in a concentration of 0 to about 6% weight per unit volume (w/v);
(b) at least about 3% weight per unit volume (w/v) carbonyl; and
(c) phenolics in a concentration of less than about 2.5 mg/ml.

13. The method of claim 12, wherein the low flavor antimicrobial liquid smoke derivative comprises carbonyl in a concentration of about 8.0 to about 12.0% weight per unit volume (w/v), and a pH of less than about 6.0.

14. The method of claim 13, wherein the low flavor antimicrobial liquid smoke derivative comprises carbonyl in a concentration of about 3.0 to about 8.0% weight per unit volume (w/v), phenolics in a concentration of less than about 1 mg/ml, and a pH of about 2.0 to about 5.5.

15. The method of claim 12, wherein the low flavor antimicrobial liquid smoke derivative comprises carbonyl of at least 10% weight per unit volume (w/v).

16. The method of claim 11, wherein the low flavor antimicrobial liquid smoke derivative is produced by processing liquid smoke through an evaporator to separate and condense the low boiling elements thereof to produce the derivative of liquid smoke.

17. The method of claim 11, wherein the oral composition further comprises at least one flavoring agent.

18. A method for inhibiting growth of a microorganism in the oral cavity of a subject, the method comprising applying an oral composition comprising an oral delivery agent and an antimicrobial agent comprising an effective amount of a low flavor antimicrobial liquid smoke derivative to the oral cavity of the subject for a time sufficient to inhibit growth of a microorganism in the oral cavity of the subject.

19. The method of claim 18, wherein the microorganism is selected from the group consisting of Streptococcus mutans, Fusobacterium nucleatum, and Porphrymonas gingivalis.

20. The method of claim 18, wherein the oral delivery agent is selected from the group consisting of a chewing gum, an edible film, a confectionary, a dentifrice, a lozenge, a mouthwash, and a mouth spray.

21. The method of claim 18, wherein the derivative of liquid smoke comprises:
(a) titratable acidity in a concentration of 0 to about 6% weight per unit volume (w/v);
(b) at least about 3% weight per unit volume (w/v) carbonyl; and
(c) phenolics in a concentration of less than about 2.5 mg/ml.

22. The method of claim 21, wherein the derivative of liquid smoke comprises carbonyl in a concentration of about 8.0 to about 12.0% weight per unit volume (w/v), and a pH of less than about 6.0.

23. The method of claim 22, wherein the derivative of liquid smoke comprises carbonyl in a concentration of about 3.0 to about 8.0% weight per unit volume (w/v), phenolics in a concentration of less than about 1 mg/ml, and a pH of about 2.0 to about 5.5.

24. The method of claim 22, wherein the derivative of liquid smoke comprises carbonyl of at least 10% weight per unit volume (w/v).

25. The method of claim 18, wherein the derivative of liquid smoke is produced by processing liquid smoke through an evaporator to separate and condense the low boiling elements thereof to produce the derivative of liquid smoke.

26. The method of claim 18, wherein the oral composition further comprises at least one flavoring agent.
